(19) United States
(12) Patent Application Publication
(10) Pub. No.: US 2019/0282759 A1
(43) Pub. Date: Sep. 19, 2019

(54) PRE-FILLED INJECTION DEVICE WITH CARTRIDGE, HAVING MULTIPLE CHAMBERS

(71) Applicant: OROFINO PHARMACEUTICALS GROUP SRL, Rome (IT)

(72) Inventor: Ernesto OROFINO, Rome (IT)

(21) Appl. No.: 16/317,575

(22) PCT Filed: Jul. 10, 2017
(86) PCT No.: PCT/IB2017/054142

§ 371 Date: Jan. 14, 2019

(30) Foreign Application Priority Data
Jul. 19, 2016 (IT) 102016000075511

(51) Int. Cl. A61M 5/28; A61M 5/24

(52) U.S. Cl. CPC A61M 5/284; A61M 5/2448; A61M 2005/2462; A61M 5/286; A61M 2005/2407; A61M 5/2459

(57) ABSTRACT

An injection device (100) including a tubular body (3) within which a cartridge is inserted (10) equipped with a bottom (14) and a back chamber (8) containing one first substance (SI). The device (100) also has a front chamber (9) containing one second substance (S2) intended to be mixed inside the tubular body (3) with the first substance (SI) to reconstitute the injectable substance (S). The device (100) is characterized in that the cartridge (10) has a flexible body (13) suitable for tearing as a result of the increase of pressure inside the cartridge (10) in order to put in communication the back chamber (8) with the front chamber (9) and to allow the reconstitution of the injectable solution (S).

The present invention relates to a device for injecting a solution reconstituted immediately before being administered. In particular, the invention refers to the technical sector of syringes of the pre-filled multiple chamber type with cartridge.

In the pharmaceutical and medical devices sector, it is necessary that the sterility of the product is guaranteed from the first production step until the use of the product thereof. Furthermore it is necessary that the sterility is guaranteed also during the reconstitution step of the solution immediately before being administered.

An example of known syringes of the pre-filled type and with cartridge is shown in document U.S. Pat. No. 3,760,503. Such syringe includes one first liquid component housed inside the body of the syringe and one second solid component housed separately inside a specific cartridge, such cartridge is as well inserted inside the body of the syringe. The two liquid and solid components are combined for reconstituting the injectable solution immediately before being administered.

In order to reconstitute the injectable solution, a specific plunger is inserted inside the syringe, firstly breaking a protective seal of the cartridge, and thereafter the bottom of the cartridge itself in order to allow the combination and the mixing of the two liquid and solid components. The bottom of the cartridge is thus perforated by the plunger.

Therefore, in order to reconstitute the injectable solution, it is necessary to insert an external perforating element (plunger) inside the syringe. Such an aspect may represent a relevant disadvantage regarding the sterility of the injectable solution: contaminating elements may in fact enter inside the syringe along with such external perforating element, thus compromising the sterility of the injectable solution. Furthermore, while perforating the bottom of the cartridge by means of the perforating external element (plunger) there is the risk of tearing jaggedly the wall of the cartridge creating hazardous detachments of material that may contaminate the injectable solution.

Therefore, the known syringe is not able to guarantee the sterility of the product even during the reconstitution step of the solution immediately before being administered. Such syringe, intended for the dental sector, is absolutely not suitable for the pharmaceutical and medical devices sector.

The need perceived in the pharmaceutical and injection device sector is making available a device enabling to guarantee the sterility of the product even during the reconstitution step of the solution immediately before being administered.

The objective of the present invention is to solve the prior art problems taking into consideration the needs of the sector.

Such objective is reached by an injection device, in particular by a syringe of the pre-filled multiple chamber type with cartridge, wherein the cartridge has a flexible body that is elastically deformable and tearable as a result of the increase of pressure inside the cartridge.

Furthermore, the solution according to the present invention is particularly advantageous as the cartridge has specific debilitations suitable for directing the breaking of the bottom of the cartridge thereof avoiding hazardous detachments of material that may contaminate the injectable solution.

The solution according to the present invention is suitable in general for injection devices and, in particular for injection devices of the pre-filled multiple chamber type.

Such objective is reached by an injection device according to claim 1. The dependent claims disclose preferred or advantageous embodiments of the device.

The characteristics and the advantages of the injection device according to the present invention will be apparent by the hereinafter description, by way of example but not limited to, according to the enclosed drawings, wherein:

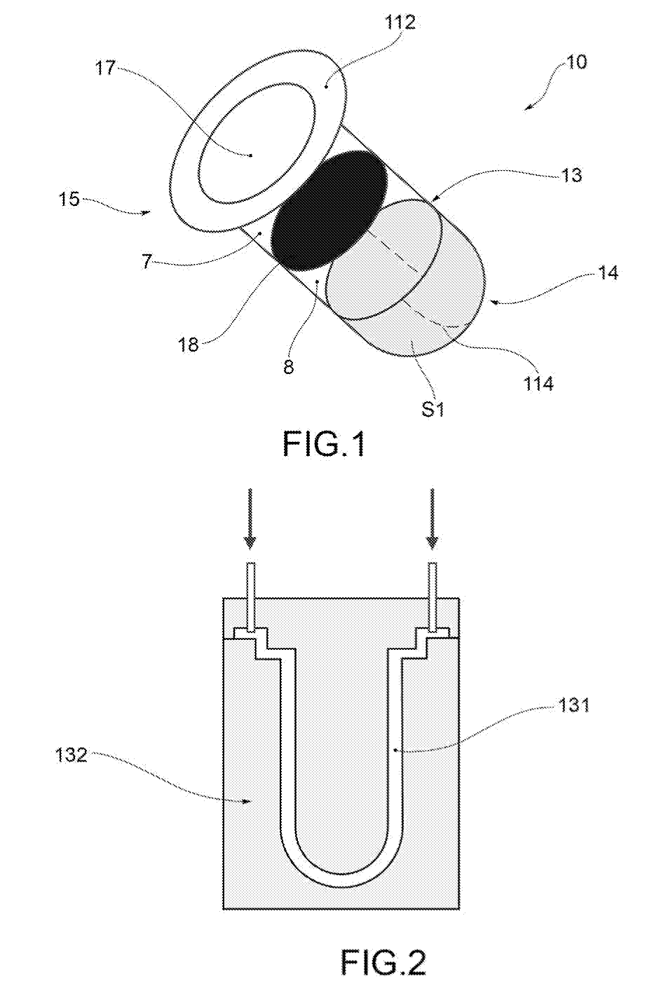

FIG. 1 shows an axonometric view of one component of the injection device according to the present invention, in particular of a pre-filled cartridge;

FIGS. 2 to 4 show some production steps of the cartridge of FIG. 1, in particular the moulding, the filling with a first component and the closure steps;

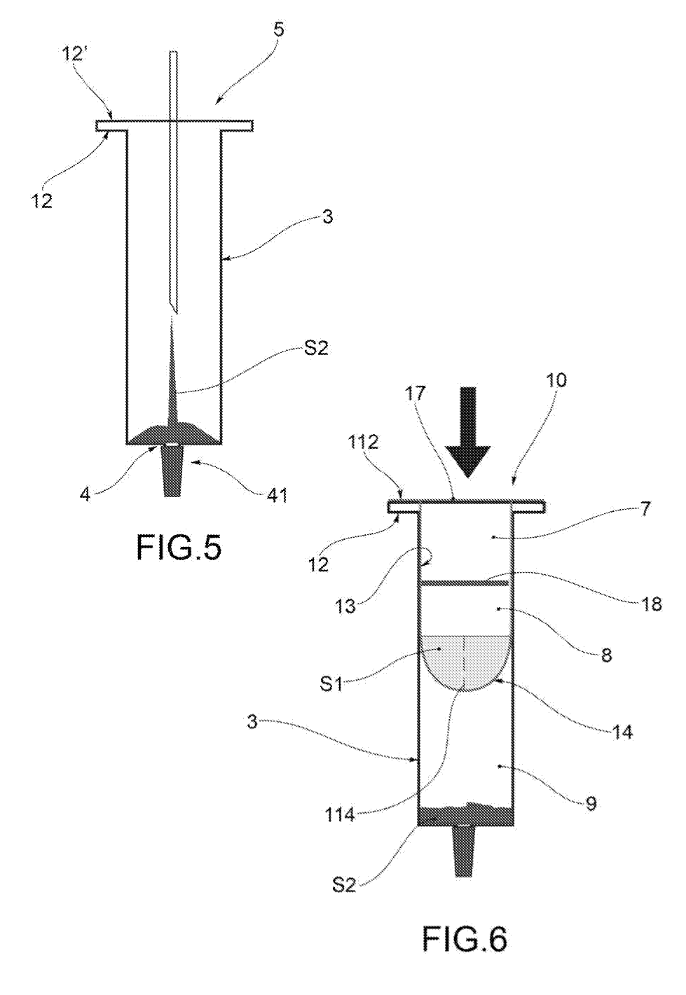

FIGS. 5 to 8 show some production steps of the injection device according to the present invention, in particular the filling step with a second component, the insertion of the cartridge of FIG. 1, the fixing of the cartridge in position, the insertion of the plunger under sterile conditions; in particular FIG. 8 shows the sterile injection device and ready for use:

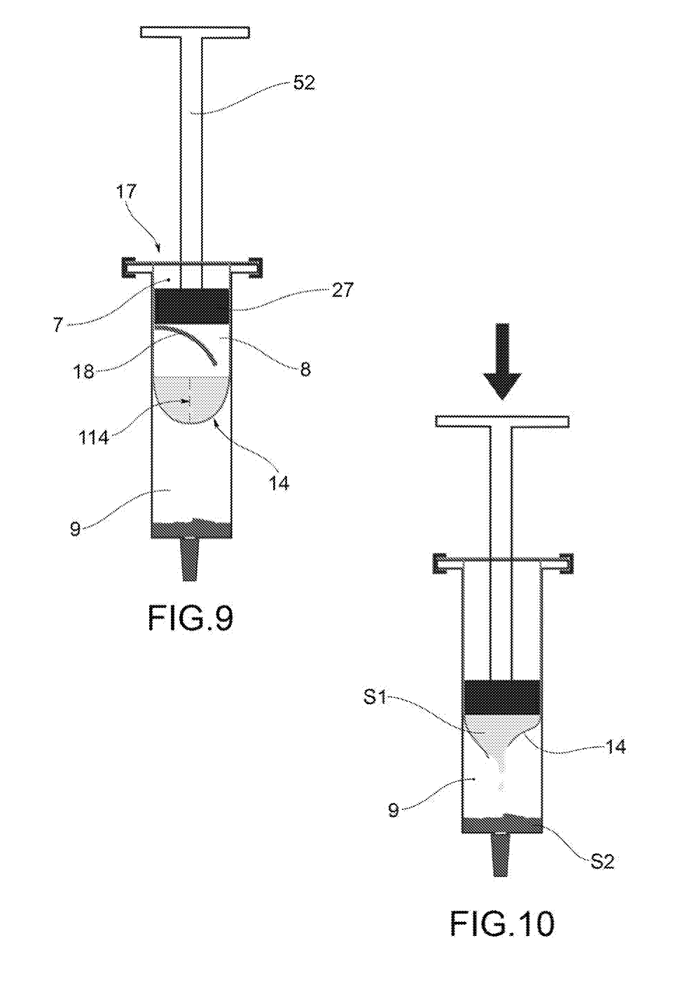

FIGS. 9 to 13 show some usage steps of the injection device according to the present invention, in particular, the breaking step of the protective seal of the cartridge, the breaking of the cartridge for combining the first and the second components, the reconstitution of the solution, the outflow of air, the injection of the solution; in particular FIG. 12 shows the sterile injection device ready for the injection.

Referring to the enclosed figures, and in particular to FIG. 8, reference number 100 indicates an injection device.

In the embodiment illustrated in the aforementioned figures, the injection device is a pre-filled, multiple chamber syringe with cartridge.

The pre-filled injection device 100 enables to reconstitute an injectable solution S immediately before being administered.

Referring to FIG. 5, the pre-filled injection device 100 includes one containment tubular body 3, extending between one first opening 4, or front opening, and one second opening 5, or back opening 5.

The tubular body 3 is the containment body of a syringe, suitable for containing injectable substances and it is preferably made of glass or of a transparent or substantially transparent plastic material. Preferably, the tubular body 3 is made in one single piece.

The front opening 4 is suitable for enabling the outflow of an injectable solution S.

The front opening 4 of the tubular body 3 is closed by a removable cap 41, suitable for sealing at the bottom the tubular body 3 until the injection device 100 is used. In particular, FIG. 8 shows the sterile injection device 100 and ready for use, wherein the front opening 4 of the tubular body 3 is closed by the removable cap 41.

Once the cap 41 is removed, the front opening 4 of the tubular body 3 is engageable with a needle 42 suitable for injecting the injectable solution. In particular FIG. 12 shows the sterile and ready for use injection device 100, equipped with a needle 42 in fluid communication with the front opening 4 of the tubular body 3.

The tubular body 3 is equipped, at the back opening 5, with an ergonomic grasp portion 12, fixed to the tubular body 3 or integrally produced with the tubular body 3. The ergonomic grasp portion 12 includes a supporting edge 12′, suitable for enabling to fix in position a cartridge 10 inserted inside the tubular body 3 of the injection device 100.

In the working configuration illustrated in FIG. 8 called “initial configuration”, the injection device 100 includes one first containment chamber 8 and one second containment chamber 9, spaced apart from each other.

The first containment chamber 8, also called back chamber, is arranged inside a cartridge 10, in turn inserted in the tubular body 3. The second containment chamber 9, also called front chamber, is arranged inside the tubular body 3.

The cartridge 10 is flexible and extendible. The cartridge 10 is made of rubbery plastic material

The cartridge 10 includes a flexible body 13.

In particular, the flexible body 13 is made of a material that is elastically deformable and tearable as a result of the increase of pressure inside the cartridge 10.

Preferably, the flexible body 13 is a latex bag, made in one single piece.

The flexible containment body 13, substantially tubular, extends from a back inlet 15 and a closed bottom 14. The flexible body 13 for example is casing-shaped.

Preferably, the flexible body 13 is closed on top, at the back inlet 15, by a breakable or a removable seal 17 to enable to open the cartridge 10.

The flexible body 13 is equipped, at the back inlet 15, with a fixing edge 112, suitable for enabling to fix the cartridge 10 in position inside the tubular body 3 of the injection device 100.

In the working configuration illustrated in FIG. 4 called “closed configuration”, the cartridge 10 includes the back containment chamber 8 and an additional empty chamber 7, each other sealingly separated by a breakable wall 18.

The empty chamber 7, also called housing 7, is defined inside the flexible body 13 between the back inlet 15 and the breakable wall 18, or between the seal 17 (if present) and the breakable wall 18.

The housing 7 is suitable for housing the sliding cap 27 linked to the plunger 52 of the injection device. In one variant embodiment not shown in the figures, the cartridge 10 has no breakable wall 18, and the back chamber 8 and the empty chamber 7 are sealingly separated between them directly by the sliding cap 27 linked to the plunger 52 of the injection device. Preferably, the flexible body 13 of the cartridge 10 has a small step 118, at the internal wall. Such small step 118 is obtained through a variation of the internal diameter of the flexible body 13. In particular, the internal diameter D at the housing 7 is greater than the internal diameter d of the back chamber 8. Advantageously, the greater diameter D at the housing 7 facilitates the insertion of the sliding cap 27 inside the cartridge 10, reducing the risk of damaging and tearing the internal walls of the flexible body 13.

Advantageously, the lesser diameter d at the back chamber 8 guarantees the sealing between the internal walls of the flexible body 13 and the sliding cap 27, thus avoiding outflows of the injectable substance S at the plunger 52.

In one not shown variant embodiment, the little step is directly formed inside the tubular body 13 of the injection device 100. In such example, the small step is obtained through a variation of the internal diameter of the flexible body 13. In particular, the internal diameter right below the grasping portion 12 is greater than the internal diameter of the front chamber 9.

The back chamber 8 is defined inside the flexible body 13 between the seal 17 and the bottom 14.

The back chamber 8 contains one first substance S1, preferably liquid.

Preferably, the bottom 14 of the cartridge 10 is equipped with at least one debilitated portion 114 suitable for directing the breaking the bottom 14. The debilitated portion 114 therefore represents a controlled breaking point of the cartridge 10 suitable for avoiding hazardous detachments of material that may contaminate the injectable solution S.

For example, the debilitated portion 114 is a pre-incision or a thinning of the flexible body wall 13. Preferably, the debilitated portion 114 is a pre-incision line extending from one end to the other of the portion that constitutes the bottom 14.

In the embodiment of FIG. 1, wherein the cartridge 10 has a casing shape with dome-shaped bottom 14, the debilitated portion 114 is a pre-incision line extending from one end to the other of the dome-shaped portion that constitutes the bottom 14.

In the embodiment of FIG. 1, the cartridge 10 includes, following the order (from the back inlet 15):

a breakable (optional) seal 17, suitable for sealingly closing the cartridge 10;
an (empty and sterile) housing 7;
an (optional) breakable wall 18;
one back chamber 8, suitable for containing one first substance S1;
one first liquid substance S1, contained inside the back chamber 8;
a bottom 14, preferably equipped with at least one debilitation 114.

The injection device 100 therefore internally includes a sterile cartridge 10 and pre-filled with one first substance S1.

Once the cartridge 10 is inserted inside the injection device 100, between the bottom 14 of the cartridge 10 and the front opening 4 (closed by the removable cap 41), inside the tubular body 3, the front chamber 9 is defined.

The front chamber 9 contains one second substance S2, preferably solid (e.g. a sterile powder).

Once the cartridge 10 is inserted into the injection device 100, inside the (empty and sterile) housing 7 of the cartridge 10 thereof, also a sliding cap 27 connected to a plunger 52 is inserted under sterile conditions.

The sliding cap 27 is therefore arranged inside the cartridge, just above the breakable wall 18 which sealingly closes the back chamber 8. The sliding cap 27 is suitable for sliding inside the cartridge 10 and the tubular body 3 of the syringe due to a pushing or pulling force exerted by the plunger 52.

The cap 27 is for example made of rubbery and/or plastic material and it is such that it firstly engages with the internal walls of the flexible body 13 of the cartridge 10 and then with the internal walls of the tubular body 3 of the injection device 100, and it is such that is can slide inside the cartridge 10 and the injection device 100 upon the pushing or pulling action of the plunger 52.

In the embodiment of FIG. 8, the injection device 100 includes, following the order (from the back wall 5):
a sliding cap 27, preferably arranged in the housing 7 of the cartridge 10, and suitable for sliding inside the cartridge 10 and the syringe due to a pushing or pulling force exerted by the plunger 52;
preferably a breakable wall 18, arranged inside the cartridge 10;
a back wall 8, defined inside the cartridge 10, suitable for containing one first substance S1;
one first liquid substance S1, contained inside the back chamber 8;
one partition wall composed by the bottom 14 of the cartridge 10, preferably equipped with at least one debilitation 114;
a back chamber 9, suitable for containing one second substance S2;
one second solid or liquid substance S2, contained in the front chamber 9, intended to be mixed inside the tubular body 3 with the first substance S1 for the reconstitution of an injectable solution S;
a front opening 4 suitable for enabling the outflow of the injectable solution S, preferably closed by the removable cap 41.

Preferably, the first liquid substance is a solvent for an injectable use, for example a WFI (Water For Injection) solvent or a lidocaine solution or a solution of water and benzyl alcohol or a saline solution of sodium chloride or in general any injectable substance suitable for reconstituting another solid or liquid substance. The first liquid substance may be or may contain an API (Active Pharmaceutical Ingredient).

Preferably, the second substance is a highly active substance.

The second substance is for example a powder, a substance in granules or a sterile tablet or a compacted powder. The aforesaid second substance may be or may contain an API. According to one embodiment, the second substance includes two separate substances for example in the form of two different tablets, each containing one of said two separate substances.

In case the second substance is solid, it may be a crystallized or freeze-dried substance. The version wherein the second substance is crystallized and not freeze-dried is presently the preferred one.

The aforesaid second substance is for example a highly active substance, as for example: an antibiotic, or a beta-lactam antibiotic (cephalosporin and/or penicillin antibiotic) or a cytotoxic anticancer or a hormone or a biological preparation or a biotechnological product, or a monoclonal antibody, or a protein, or a vaccination, or an anaesthetic. The aforesaid second substance may also be a common active substance, that is not definable as a highly active substance.

FIGS. 2 to 4 show some production steps of the cartridge of FIG. 1.

FIG. 2 shows the step of moulding the flexible body of the cartridge 10: in an injection moulding machine 132, the rubbery and/or plastic material is injected inside the moulding cavity 131 suitable for defining the casing shape of the flexible body 13. The flexible body 13 is produced under sterile conditions. Preferably, the bottom 14 of the flexible body 13 of the cartridge 10 is debilitated, for example through pre-incisions 114.

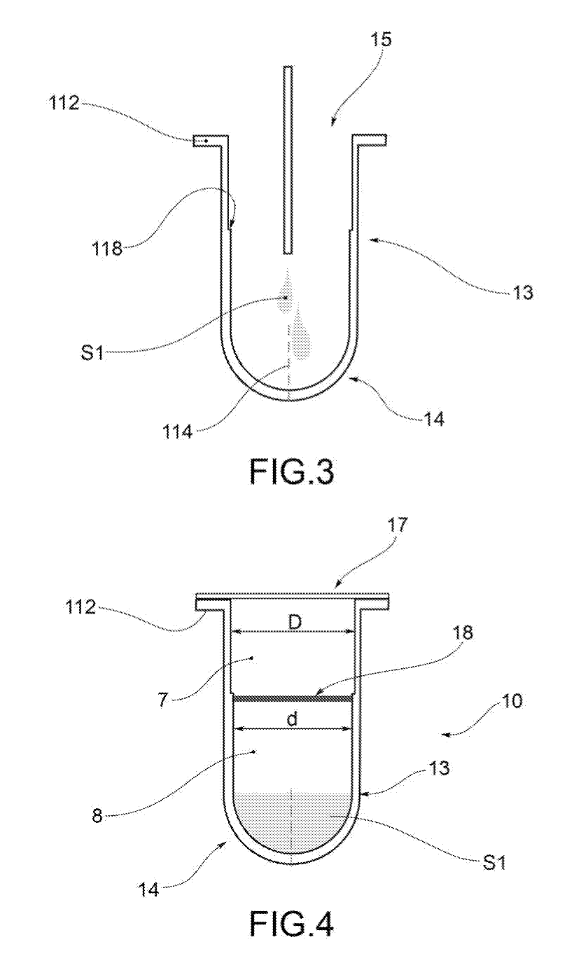

FIG. 3 shows the step of filling the cartridge 10: one first substance S1, e.g. a sterile diluent is dosed and inserted in a production machine kept under sterile conditions, inside the flexible body 13 (through the back inlet 15).

FIG. 4 shows the step of closing the cartridge 10: in the same production machine the breakable wall 18 suitable for sealingly closing the back chamber 8 is firstly inserted through the back inlet 15 and then fixed inside the flexible body 13, for example by heat sealing. Preferably, the seal 17 suitable for sealingly closing the housing 7 too is then fixed at the fixing edge 112.

FIG. 4 thus shows one sterile cartridge 10 according to the present invention.

FIGS. 5 to 8 show some production steps of the injection device 100.

FIG. 5 shows the step of filling the injection device 100: one second substance S2, e.g. a sterile powder, is dosed and inserted (through the back opening 5) inside the tubular body 3 (kept with the front opening 4 downwardly oriented and closed by the removable cap 41) in a production machine kept under sterile conditions.

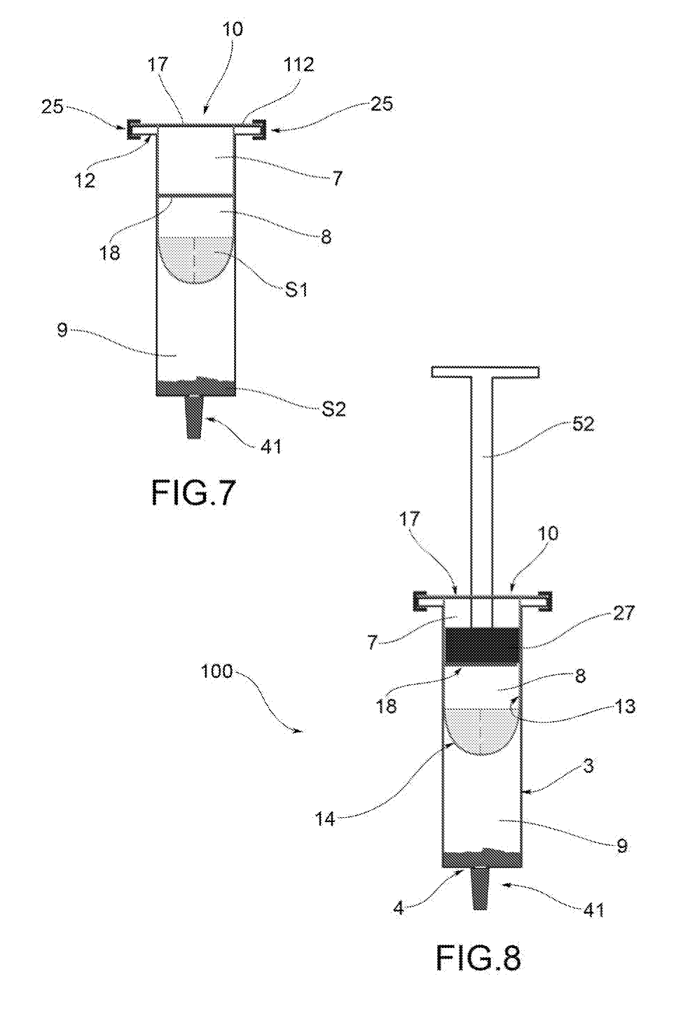

FIGS. 6 and 7 show the steps of assembling the cartridge 10 in the injection device 100.

In particular FIG. 6 shows the step of inserting the cartridge 10 inside the tubular body 3 of the injection device 100: the bottom 14 of the cartridge 10 is inserted in the back opening 5 of the tubular body 3, and the cartridge 10 is slided within the tubular body 3 until the fixing edge 112 of the cartridge 10 abuts against the supporting edge 12′ (defined by the ergonomic grasp portion 12) of the tubular body 3 of the injection device 100.

In particular FIG. 7 shows the step of fixing in position the cartridge 10. The fixing is obtained through the engagement of the fixing edge 112 of the cartridge 10 with the supporting body 12′ of the injection device 100.

In one embodiment such fixing is obtained by gluing or heat sealing.

In one different embodiment such fixing is obtained mechanically, for example through clips 25. Therefore in such example the edge 112 of the cartridge 10 and the edge 12 of the injection device are overlapped and clamped by a snap-fit clip 25, e.g. made of plastic.

FIG. 8 shows the step of inserting the plunger 52 under sterile conditions; the sliding cap 27 is inserted from the back inlet 15 of the flexible body 13 and is slided in the cartridge 10 until it abuts against the breakable wall 18.

In case the cartridge 10 has a breakable seal 17, such seal is removed before inserting the sliding cap 27, or it is torn to enable to insert the sliding cap 27.

Once the sliding cap 27 is inserted, the plunger 52 is fixed (for example screwed) to the cap 27 and the injection device 100 is sterile and is ready to be sold and used.

In one variant, the plunger 52 is fixed to the cap 27 before being inserted in the cartridge 10.

Advantageously therefore, the sliding cap 27 and the plunger 52 are inserted inside the injection device 100 directly during the production step under sterile conditions.

First of all this allows to guarantee the sterility of the device 100 thereof, as contaminant elements are prevented from entering inside the housing 27 of the cartridge 10 together with the cap 27 or the plunger 52 compromising the sterility of the injectable solution S.

Furthermore, the step of inserting the sliding cap inside the housing 27 of the cartridge 10 is a very delicate operation: by the implementation of such operation directly during the production step (thus performed by skilled personnel or by high precision machinery) it is avoided breakings and tearings of the flexible body 13 of the cartridge 10 that may lead to leakages of the injectable solution or to an earlier reconstitution of the injectable solution making the injection device 100 unusable.

FIGS. 9 to 13 show some production steps of the injection device 100.

Starting from the initial configuration of FIG. 8, due to a pushing action of the plunger 52, e.g. due to a manual driving of the plunger 52, the sliding cap 27 pushes against the breakable wall 18 until it breaks it, thus reaching a pre-reconstitution configuration wherein, thanks to the still intact bottom 14 of the cartridge 10, the back chamber 8 and the front chamber 9 are still sealingly separated from each other. By further pushing the plunger 52 (FIG. 10), the further advancement of the plunger 27 pushes the air and the first substance S1 inside the chamber 8 deforming the flexible body 13 of the cartridge 10 until it breaks the bottom 14 tearing it. In fact, the flexible body 13 is made of a material that is elastically deformable and tearable as a result of the increase of pressure inside the back chamber 8. The cap 27 advances inside the back chamber 8 reducing the volume and determining a pressure increase; as a result of the increase of pressure inside the back chamber 8 the flexible body 13 deforms: once reached the maximum breaking load of the material composing the flexible body 13, the cartridge tears at the bottom 14.

Advantageously, the debilitated portion 114 represents the trigger point of the bottom breaking 14, thus ensuring one controlled tearing with no unravelling o detachment of the material composing the flexible body 13.

Once broken the bottom 14 of the cartridge 10, the back chamber 8 and the front chamber 9 are in fluid communication, and the first substance S1 contacts the second substance S2 to mix with it.

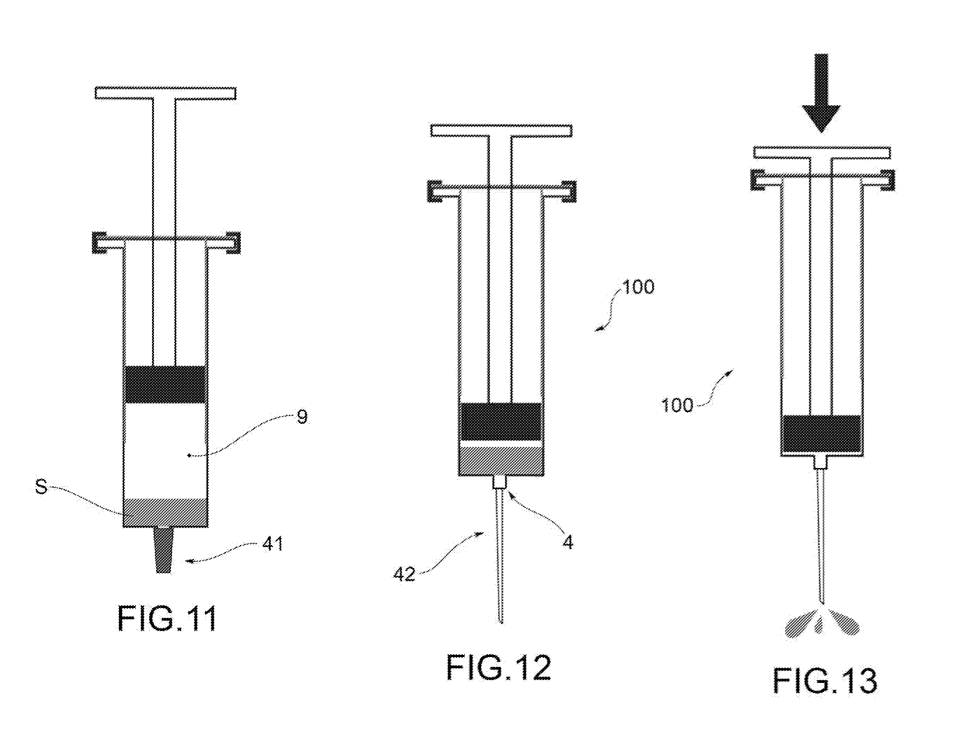

Once broken the bottom 14 of the cartridge 10, one so called reconstitution configuration is reached (FIG. 11). In the aforesaid reconstitution configuration, shaking for example manually the injection device 100 may promote the mixing of the two substances.

At this point, it is necessary to eject the air contained in the front chamber 9. It is therefore necessary to overturn the injection device 100 orienting upwardly the front opening 4 closed by the removable cap 41, remove the cap 41 and further push the plunger 52 advancing the cap 27 in order to reduce the volume of the front chamber 9 and eject the air contained thereof from the tubular body 3 through the opening.

Once the needle 42 is applied to the front inlet 4, the sterile injection device 100 is ready for the injection.

By further pushing the plunger 52 (FIG. 13) it is possible to advance the cap 27 to reduce the volume of the front chamber 9 and eject the reconstituted solution S from the tubular body 3 through the opening 4.

Innovatively, an injection device of the pre-filled multiple chamber type with cartridge according to the present invention is able to guarantee the sterility of the injectable solution even during the step of reconstituting the solution immediately before being administered.

Advantageously in fact, as the cartridge has a flexible body that is elastically deformable and tearable due to the mere increase of pressure inside the cartridge, no perforation element of the cartridge itself is necessary to reconstitute the solution. In fact, the cartridge opens by itself (bursts) as a result of the increase of pressure inside the back chamber.

Furthermore advantageously, as the cartridge has specific debilitations suitable for directing the breaking of the cartridge thereof, hazardous detachments of material that may contaminate the injectable solution are avoided.

Advantageously, as the pushing cap is pre-inserted in the injection device during the production step under sterile conditions, contaminant elements are prevented from entering inside the device thereof compromising the sterility of the solution.

Advantageously, as such step of inserting the pushing cap is a very delicate operation, the implementation during the production step avoids damages that may lead to leakages of the solution or to an earlier reconstitution making the injection device 100 unusable.

Subject to the principle of the invention, its embodiments and implementation details shall be widely varied with respect to what described and illustrated for exemplary purposes but not limited to, without departing from the scope of protection as defined in the enclosed claims.

1. A pre-filled injection device, for injecting one injectable solution reconstituted immediately before being administered, including:
one containment tubular body extending between one front opening for the outflow of the injectable solution, and one back opening;
one cartridge at least partially arranged inside the tubular body, said cartridge having a bottom and a back chamber containing one first substance;
one front chamber, defined inside the tubular body between the front opening and the bottom of the cartridge, said front chamber containing one second substance intended to be mixed inside the tubular body with the first substance for the reconstitution of the injectable solution;
wherein:
the cartridge is flexible and extendible and has a flexible body suitable for tearing as a result of the increase of pressure inside the cartridge in order to put in communication the back chamber with the front chamber and to allow the reconstitution of the injectable solution;
the flexible body is made of elastically deformable material;
the flexible body is substantially tubular and extends from a back inlet and a closed bottom;
the pre-filled injection device comprises a sliding cap arranged inside the cartridge and suitable for sliding inside the cartridge.

2. The injection device, according to claim 1, wherein the closed bottom is dome shaped.

3. The injection device, according to claim 1, wherein the flexible body is a latex bag.

4. The injection device according to claim 1, wherein the closed bottom of the cartridge has at least one debilitated portion suitable for directing the breaking of the cartridge.

5. The injection device, according to claim 4, wherein the debilitated portion is a pre-incision line extending from one to the other end of the closed bottom.

6. The injection device, according to claim 1 wherein the sliding cap is configured to determine the increase of pressure inside the cartridge.

7. The injection device, according to claim 6, wherein the cartridge has a housing defined between a back inlet and the back chamber, and wherein said sliding cap is arranged inside the housing of the cartridge.

8. The injection device, according to claim 7, wherein the back chamber and the housing are sealingly separated from the sliding cap.

9. The injection device, according to claim 7, wherein the back chamber and the housing are each other sealingly separated by a breakable wall, and wherein the sliding cap is suitable for breaking said wall.

10. The injection device, according to claim 7, wherein the cartridge is upperly closed, at the back inlet, by one seal, breakable or removable, to allow to insert the sliding cap.

11. The injection device, according to claim 1, wherein the cartridge has, at the back inlet, a fixing edge to fix the cartridge in position inside the tubular body, said fixing being implemented at a supporting edge arranged at the back inlet of the tubular body.

12. The injection device, according to claim 1, wherein the internal wall of the flexible body or of the tubular body has a small step obtained through a variation of the internal diameter of the flexible body.

13. The injection device, according to claim 11, wherein the internal diameter, of the flexible body or of the tubular body, at the housing is greater than the internal diameter at the back chamber.

14. A flexible cartridge that can be inserted in an injection device, including:
one back inlet and, on the opposite side, one closed bottom;
one breakable wall suitable for separating a housing from a back chamber;
one housing, defined between the back inlet and the wall;
one back chamber, defined between the wall, containing one first substance;
wherein the cartridge is extendible and is composed by a flexible body suitable for tearing as a result of the increase of pressure inside the back chamber,
wherein the flexible body is substantially tubular and extends from the back inlet and the closed bottom.

15. The flexible cartridge, according to claim 14, wherein the closed bottom has at least one debilitation suitable for directing the breaking of the cartridge.
